(19) United States
(12) Patent Application Publication
(10) Pub. No.: US 2026/0330629 A1
(43) Pub. Date: Oct. 22, 2026

(54) SYSTEM FOR DETECTING AND ANALYZING POSITION OF TUBES AND CATHETERS WITHIN HUMAN BODY USING DEEP LEARNING-BASED DUAL-STAGE MODEL

(71) Applicants: ZIOVISION CO.,LTD., Chuncheon-si, Gangwon-do (KR); CHUNG ANG UNIVERSITY INDUSTRY ACADEMIC COOPERATION FOUNDATION, Seoul (KR)

(72) Inventors: Hyun-Soo CHOI, Chuncheon-si, Gangwon-do (KR); Inseo PARK, Chuncheon-si, Gangwon-do (KR); Gwiseong MOON, Chuncheon-si, Gangwon-do (KR); Ji Young HONG, Chuncheon-si, Gangwon-do (KR); Hongseok KO, Chuncheon-si, Gangwon-do (KR); Doohee LEE, Chuncheon-si, Gangwon-do (KR); Yoon KIM, Chuncheon-si, Gangwon-do (KR); Woo Jin KIM, Chuncheon-si, Gangwon-do (KR); Kyoung Min MOON, Gangneung-si, Gangwon-do (KR); Jeongwon HEO, Chuncheon-si, Gangwon-do (KR)

(73) Assignees: ZIOVISION CO.,LTD., Chuncheon-si, Gangwon-do (KR); CHUNG ANG UNIVERSITY INDUSTRY ACADEMIC COOPERATION FOUNDATION, Seoul (KR)

(21) Appl. No.: 19/100,578

(22) PCT Filed: Oct. 23, 2024
(86) PCT No.: PCT/KR2024/016212

§ 371 Date: Feb. 3, 2025

(30) Foreign Application Priority Data
Oct. 24, 2023 (KR) 10-2023-0142918
Oct. 23, 2024 (KR) 10-2024-0145854

(51) Int. Cl. G06T 7/00; G06T 5/50; G06T 7/12; G06V 10/764; G06V 10/776; G16H 30/40

(52) U.S. Cl. CPC G06T 7/0012; G06T 5/50; G06T 7/12; G06V 10/764; G06V 10/776; G16H 30/40; G06T 2207/10116; G06T 2207/20221; G06T 2207/30021

(57) ABSTRACT

The present invention relates to a system for detecting and analyzing a position of an intubation tube within a human body including a segmentation deep learning module that receives an X-ray image and generates an intubation tube mask for a position of an intubation tube intubation within a human body included in the X-ray image, a concatenation module that connects the intubation tube mask and the X-ray image to generate a mask-combined X-ray image, and a classification deep learning module that receives the mask-combined X-ray image and classifies appropriateness of the position of the intubation tube within the human body.

### TECHNICAL FIELD

The present invention relates to a system for detecting and analyzing a position of an intubation tube in a human body using a deep learning-based dual-stage model.

### BACKGROUND ART

Intubation within the human body is a medical practice essential for the treatment and maintenance of life of patients, and is used for various purposes such as nutrition supply, medication administration, body fluid removal, etc. Intubation devices for intubation within the body includes nasogastric tubes, central venous catheters, endotracheal tubes, thoracic drainage tubes, etc. These various intubation devices should be inserted into a correct position according to their respective purposes, and when the insertion position thereof is incorrect, serious complications may occur.

For example, a nasogastric tube is mainly used to provide nutrition or administer medication to a patient, and if the nasogastric tube is incorrectly inserted into the airway or does not reach a correct position due to its incorrect insertion position, it can cause serious complications such as pneumonia, pneumothorax, tracheal perforation, etc.

An existing method of checking the position of the nasogastric tube relies on the manual work of medical staff directly analyzing a chest X-ray image, and an error that occurs during this process may be fatal to patient safety. In this existing method, a misdetermination may occur, in particular, due to factors such as fatigue of the medical staff, lack of experience, or low resolution of the chest X-ray, which may result in cases where incorrect insertion of the nasogastric tube is not detected. Due to the limitations of this manual work method, the need for more accurate and automated techniques for checking positioning of the nasogastric tube is emerging. An automated system may reduce the burden on medical staff, increase the accuracy of checking of the positioning, and significantly improve patient safety.

Recently, various methods utilizing deep learning have been introduced to check the position of the nasogastric tube. There have been attempts to automatically detect the position of nasogastric tubes, central venous catheters, and endotracheal tubes using neural network architectures such as DenseNet, Inception-v3, and EfficientNet.

However, these approaches mainly focused on classification, and had limitations in visually explaining or interpreting the position of the nasogastric tube. A classification model usually probabilistically indicates whether a specific feature (e.g., incorrect insertion) is present, but do not provide spatial information about an exact position or path of the tube. Due to these limitations, medical staff may have difficulties in clinically interpreting and utilizing the results provided by the model. For example, since it is difficult to identify exactly where the nasogastric tube was incorrectly inserted with the classification result alone, it is insufficient to apply the classification result to clinical decision-making.

Ultimately, there is a need for a new method that can help determine whether intubation tubes for intubation within the body including nasogastric tubes have been properly inserted.

### DISCLOSURE OF THE INVENTION

### Technical Problem

An object of the present invention is to provide a system that can visually clearly indicate an exact position of an intubation tube within the human body and automatically determine whether it has been inserted incorrectly, thereby helping medical staff make clinical decisions on whether intubation within the human body has been performed appropriately.

Meanwhile, other unspecified objects of the present invention will be additionally considered within the scope that can be easily inferred from the detailed description below and its effects.

### Technical Solution

In order to achieve the objects proposed above, the following solutions are proposed.

A system for detecting and analyzing a position of an intubation tube within a human body according to an embodiment of the present invention includes a segmentation deep learning module that receives an X-ray image and generates an intubation tube mask for a position of an intubation tube intubation within a human body included in the X-ray image, a concatenation module that connects the intubation tube mask and the X-ray image to generate a mask-combined X-ray image, and a classification deep learning module that receives the mask-combined X-ray image and classifies appropriateness of the position of the intubation tube within the human body.

In an embodiment, the intubation tube may be at least one selected from a group consisting of a nasogastric tube, a central venous catheter, an endotracheal tube, and a thoracic drainage tube.

In an embodiment, the intubation tube mask may be a mask fora shape and position of a line of the intubation tube.

In an embodiment, the intubation tube mask may further include a mask for a shape and position of a tip of the intubation tube.

In an embodiment, the mask-combined X-ray image may be generated by stacking the intubation tube mask and the X-ray image in a channel form.

In an embodiment, the system may further include a visualization module that visualizes a determination result of the classification deep learning module, and the visualization module may generate a heat-map indicating an area of evidence considered important as a basis for the determination of the classification deep learning module and display the heat-map by overlapping the heat-map on the mask-combined X-ray image.

In an embodiment, the system may further include a validation model that validates an appropriateness of the determination result of the classification deep learning module, and the validation model may output the determination result of the classification deep learning module as inappropriate when the area of evidence and the intubation tube mask do not overlap.

### Effects of the Invention

In the system for detecting and analyzing the position of the intubation tube within the human body according to an embodiment of the present invention, first, the segmentation deep learning module receives an X-ray image as input, generates an intubation tube mask including the position of the intubation tube, and combines the intubation tube mask with an original X-ray image to generate a mask-combined X-ray image. The mask-combined X-ray image is analyzed by the classification deep learning module to determine the appropriateness of the intubation. That is, by inputting an image obtained by combining spatial information about the position and shape of the intubation tube rather than simply inputting the X-ray image, the accuracy of the position determination is greatly improved compared to a simple classification model.

In addition, in the system for detecting and analyzing the position of the intubation tube within the human body according to the embodiment of the present invention, using a validation model, the reliability of the determination result can be further improved by determining whether the basis for determining the appropriateness of intubation is related to the intubation.

Finally, the system for detecting and analyzing the position of the intubation tube within the human body according to the embodiment of the present invention can contribute to medical staff making more accurate clinical determinations by visually providing the results determined by the system for detecting and analyzing the position of the intubation tube within the human body.

Meanwhile, it is added that even if the effects are not explicitly mentioned herein, the effects described in the following specification and the tentative effects thereof, as expected by the technical features of the present invention, are treated as described in the specification of the present invention.

### BRIEF DESCRIPTION OF THE DRAWINGS

FIG. 1 is a schematic diagram of a system for detecting and analyzing a position of an intubation tube within a human body according to an embodiment of the present invention.

FIG. 2 is a schematic flowchart illustrating a workflow of segmentation, connection, and classification of a system for detecting and analyzing a position of an intubation tube within a human body according to an embodiment of the present invention.

FIG. 3 is a reference diagram illustrating a process of detecting and analyzing a position of an intubation tube within a human body from an external validation dataset by the system for detecting and analyzing the position of the intubation tube within the human body according to the embodiment of the present invention.

FIG. 4 is a prototype user interface of a system for detecting and analyzing a position of an intubation tube within the human body according to an embodiment of the present invention.

The accompanying drawings are intended as reference for understanding the technical idea of the present invention, and are not intended to limit the scope of the present invention.

### MODE FOR CARRYING OUT THE INVENTION

Hereinafter, with reference to the drawings, a configuration of the present invention guided by various embodiments of the present invention and the effects resulting from the configuration will be described. In describing the present invention, when it is determined that the relevant known functions may unnecessarily obscure the gist of the present invention as they are obvious to those skilled in the art, the detailed description thereof will be omitted.

A term “module” used in this document may include a unit implemented by hardware, software, or firmware, and may be used interchangeably with terms such as logic, logic block, component, circuit, etc., for example. The module may be a component that is configured integrally or a minimum unit of the component that performs one or more functions or a part thereof.

In this document, a “module” or “node” performs tasks such as moving, storing, and converting data using a computing device such as a CPU or an AP. For example, a “module” or “node” may be implemented as a device such as a server, a PC, a tablet PC, or a smartphone.

Hereinafter, a structure, operation, and effect of a system for detecting and analyzing a position of an intubation tube within a human body according to an embodiment of the present invention will be described with reference to the drawings.

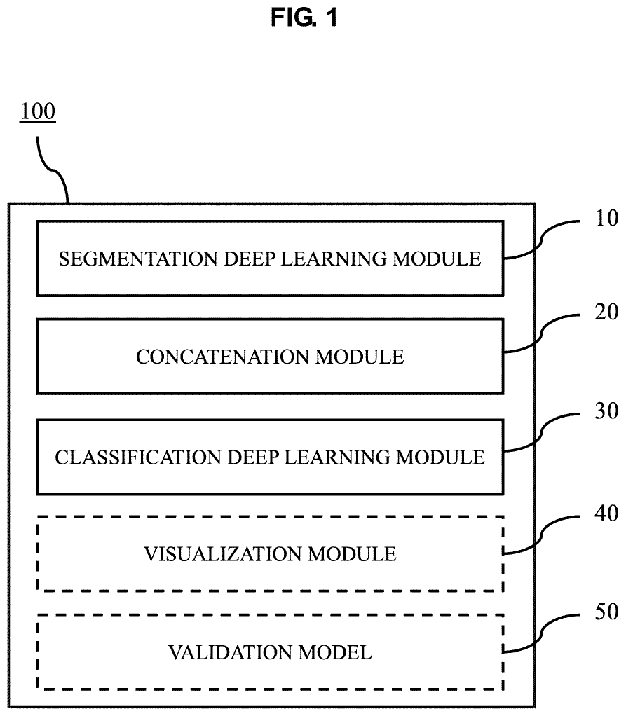

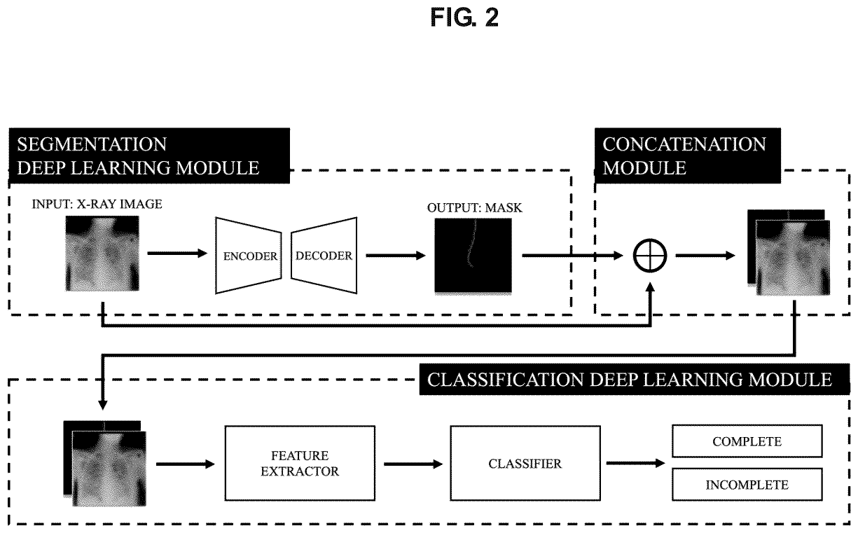

FIG. 1 is a schematic diagram of a system for detecting and analyzing a position an intubation tube within the human body according to an embodiment of the present invention, and FIG. 2 is a schematic flowchart illustrating a workflow of segmentation, connection, and classification of a system for detecting and analyzing a position of an intubation tube in the human body according to an embodiment of the present invention.

A system for detecting and analyzing a position of an intubation tube within a human body 100 according to an embodiment of the present invention is configured with three main modules, and has an integrated structure that may accurately detect and analyze the position the intubation tube by receiving an X-ray image of a patient who has undergone intubation within the human body. The three main modules may include a segmentation deep learning module 10, a concatenation module 20, and a classification deep learning module 30. Furthermore, the system for detecting and analyzing the position of the intubation tube within the human body 100 according to the embodiment of the present invention may further include a visualization module 40 and a validation model 50.

The segmentation deep learning module 10 identifies and separates a position and shape of the intubation tube, more specifically, a line of the intubation tube, in the X-ray image to generate an intubation tube mask. Furthermore, it is also possible to identify and separate a tip (end) of the intubation tube to additionally generate an intubation tube mask related to the tip. The mask means a binary image created to visually identify the position of the intubation tube in the X-ray image. For example, the mask serves to visually distinguish the position and shape of the intubation by displaying pixels where the intubation tube is placed as 1 (or a color that is visually easy to check, such as white or red) and the other parts as 0 (or black). In the system for detecting and analyzing the position of the intubation tube within the human body 100 according to an embodiment of the present invention, the intubation tube mask may display the position and shape of the line of the intubation tube to increase the accuracy of determining whether the intubation tube has been properly inserted into the human body.

The segmentation deep learning module 10 can use a deep learning model trained to determine whether each pixel corresponds to the intubation tube or not based on a pre-trained dataset. For example, nnU-Net may be used as the segmentation deep learning module 10, but the present invention is not limited thereto.

Pre-training may be performed to improve the performance of the segmentation deep learning module 10. For example, pre-training of the segmentation deep learning module 10 may be performed using the RANZCR CLIP dataset containing various information related to chest X-ray images.

In addition, k-fold cross-validation may be used to enhance the performance of the segmentation deep learning module 10. K-fold cross-validation splits the dataset into k folds, uses one of the folds as a validation dataset, and uses the remaining (k−1) folds as a training dataset. This process is repeated k times, so that each fold is used for validation once. In each repetition, each model is trained. The test set is evaluated using k trained models, and the final performance is calculated by combining the results of the k models. The method of combining the final results may use the average, but the present invention is not limited to this. Through this, the entire data may be utilized as efficiently as possible, and the overfitting problem of the model may be reduced and the generalization performance may be improved.

Finally, an ensemble strategy may be used to enhance the performance of the segmentation deep learning module 10. The ensemble is a method of combining multiple nnU-Net models to make a final prediction, and has the effect of improving the prediction accuracy by complementing for weaknesses between models.

The intubation tube mask generated by the segmentation deep learning module 10 and the original X-ray image are input into the concatenation module 20. The connection module 20 stacks the intubation tube mask and the original X-ray image in a channel form to generate a mask-combined X-ray image.

Existing technology for checking positioning of the intubation tube have mainly focused on classification methods. For example, neural networks such as DenseNet, Inception-v3, and EfficientNet have been used to probabilistically classify whether the intubation tube is placed in a malposition in the X-ray image. In order to increase classification accuracy in a deep learning model, clarity of contextual information and features is very important. However, when probabilistically classifying whether the intubation tube is placed in the malposition using only X-ray images as before, contextual information inevitably becomes ambiguous and features become unclear. The mask-combined X-ray image is a binary image that has been processed to clearly distinguish a specific area of interest, that is, the position of the intubation tube. That is, the mask-combined X-ray image allows the classification deep learning module 30 to focus on a part where the intubation tube is placed, thereby reducing interference from unnecessary information. Furthermore, since the mask-combined X-ray image is provided by combining the contextual information of the original image (surrounding tissue, positional relationship, etc.) and a segmented intubation tube mask, the model may determine a correct position of the intubation tube along with the surrounding structure, thereby deriving a more accurate classification result than a simple mask or a simple original image.

This mask-combined X-ray image is transmitted to the classification deep learning module 30 to determine whether the intubation tube inserted correctly. More specifically, the classification deep learning module 30 classifies appropriateness of the intubation through input obtained by combining the contextual information of the X-ray image and spatial information of the intubation tube mask. The classification deep learning module 30 serves to determine whether the intubation tube was inserted correctly based on features learned from an input image. This process follows the principle that a deep learning model learns patterns of classes (correctly inserted cases and incorrectly inserted cases) through a large amount of data and predicts which class a new image belongs to. The model analyzes various features (e.g., the shape, position, texture, etc.) of an image to determine whether the intubation tube is placed in a normal position and classifies it probabilistically.

As the classification deep learning module 30, a deep learning model trained to determine whether the intubation tube was inserted correctly based on a pre-trained dataset may be used. For example, ResNet50 may be used as the classification deep learning module 30, but the present invention is not limited thereto.

Pre-training may be performed to enhance the performance of the classification deep learning module 30. For example, pre-training may be performed with MedCLIP, which is the latest medical vision-text pre-training model.

Furthermore, the system for detecting and analyzing the position of the intubation tube within the human body 100 according to an embodiment of the present invention may further include the visualization module 40 and the validation model 50.

The visualization module 40 generates a heat-map indicating an area of evidence considered important as a basis when determining the classification deep learning module and displays the heat map by overlaying it on the mask-combined X-ray image. Areas considered important for determination on the heat map are indicated in red (bright areas), and areas that are not are indicated in blue (dark areas). The area of evidence may be selected as an area having a user-specified criterion value or more, and for example, it is also possible to set the green to red area as the area of evidence. In this case, the heat map may be generated using the Gradient-weighted Class Activation Mapping (Grad-CAM)) technique. Grad-CAM operates in a way that visually emphasizes an area of an image that the model considers most important when performing classification. Specifically, Grad-CAM combines a feature map of the last convolutional layer and a gradient of the corresponding class to highlight a part of the image that the model focused on when predicting a specific class. Through the mask-combined X-ray image with overlapping heat maps, the medical staff may intuitively understand the basis for the model's prediction.

The validation model 50 outputs the determination result of the classification deep learning module as inappropriate when the area of evidence and the intubation tube indicated in the intubation tube mask do not overlap each other. When the important area of evidence that the model used to make predictions does not match a position where the intubation tube should actually be placed, there is a significant possibility that the model made a decision based on a wrong criterion. Therefore, by validating whether the area of evidence and the intubation tube mask overlap, it is possible to check whether the model's prediction is reliable, that is, whether the model made a determination based on correct information, and to filter out incorrect determinations, thereby increasing the reliability of the prediction.

Hereinafter, the position detection and analysis system according to an embodiment of the present invention will be described in more detail through actual position detection and analysis of a nasogastric tube.

An example of position detection and analysis of the nasogastric tube was performed in accordance with the Declaration of Helsinki and was approved by the ethics committees of Chuncheon Sacred Heart Hospital, Gangneung Asan Hospital, and Kangwon National University Hospital. According to approval by the ethics committee, consent for anonymously using clinical data for research purposes was not required from the participants.

Data was collected as follows. In this example, a comprehensive dataset containing 2,627 anonymous patient X-ray images obtained from three major medical institutions of Chuncheon Sacred Heart Hospital, Gangneung Asan Hospital, and Kangwon National University Hospital was collected. The data obtained from Kangwon National University Hospital was used for external validation purposes. The systematic collection of these records was conducted in strict compliance with the approval of the Institutional Review Board (IRB) of each hospital. All images were carefully extracted and stored in DICOM format using a picture archiving and communication system (PACS). Because the data has undergone thorough de-identification measures, patient consent is not required. The characteristics of patients included in the dataset are summarized in Table 1 below.

TABLE 1

| | | Training & | Internal | External | |
|---|---|---|---|---|---|
| | | Validation | Testing | Testing | |
| Variable | Category | Dataset | Dataset | Dataset | P-value |
| Total (n) | — | 1,799 | 450 | 378 | — |
| Label (n) | Complete | 1,566 | 391 | 314 | 0.12 |
| | incomplete | 233 | 59 | 64 | |
| Sex (n) | Male | 974 | 275 | — | 0.77 |
| | Female | 600 | 175 | — | |
| | Unknown | 225 | — | — | |
| Age (years) | — | 77.50 ± 11.20 | 77.51 ± 10.96 | — | 0.98^{†} |
| Manufacturer | Canon | 608 | 376 | — | 0.0001 |
| (n) | DongKang | 582 | 166 | — | |
| | DK Medical Systems | 156 | 44 | — | |
| | Samsung | 66 | 21 | 172 | |
| | SIEMENS | — | — | 158 | |
| | Other | 387 | 43 | 48 | |

Values with plus/minus signs represent mean ± standard deviation. Age values from external test set are not provided because they were anonymized before data collection. For categorical variables (e.g., label, gender, manufacturer), χ² test was used, and for age, analysis of variance (denoted by ±) was used.

To obtain label data, clinical experts labeled normal insertion of NG tube (nasogastric tube) as ‘complete’ and abnormal state as ‘incomplete’. This was based on the opinions of four qualified experts (MKM, KHG, HJY, HJW). Through this process, 1,957 complete samples and 292 incomplete samples were used for model training and testing. Three respiratory specialists (MKM, HJY, HJV) and one radiologist (KHS) with more than 10 years of clinical experience collected a dataset of 2,249 chest X-rays with labels for NG tube positions. All cases were classified as ‘complete’ or ‘incomplete’.

A case where medication administration or enteral nutrition was possible through the NG tube clinically was classified as ‘complete’, and this was defined as a normal NG tube position. In contrast, a case where medication administration or enteral nutrition was impossible was classified as ‘incomplete’ and, this was defined as abnormal NG tube position. The normal tube position corresponds to a case where the NG tube does not enter the airway and the tip thereof is placed at least 3 cm below the diaphragm on the chest X-ray PA or AP view. When it was difficult for one clinical expert to classify the case as ‘complete’ or ‘incomplete’, four experts simultaneously reviewed the NG tube position on the chest X-ray. Labels were assigned according to the classification that all experts agreed on.

First, an intubation tube mask for the nasogastric tube was generated. To do this, a dedicated segmentation model fθ, which is designed to identify the intubation tube mask including the line and tip of the intubation tube in the X-ray image, was introduced. The model fθ generates a mask of a line (mₗ∈{0, 1}^{HXW}) and a tip (mₜ∈{0, 1}^{HXW}) from a preprocessed and normalized image x. Here, according to a sample index i, it is calculated as Formula 1 below.

mₛ∈{l,t}=argmax(φ(fθ(xᵢ))) [Formula 1]

Here, φ represents the postprocessing of nnU-Net. In this process, appropriate information related to the line of the nasogastric tube is extracted to prepare for improved shape recognition in subsequent steps.

Precise nasogastric tube detection was achieved by using nnU-Net, which is a state-of-the-art framework based on the U-Net architecture for biomedical segmentation, as the segmentation model fθ. To improve the accuracy of the segmentation model, a pre-training technique was strategically used. Specifically, the model fθ was underwent pre-training on RANZCR CLiP.

The original X-ray image and the intubation tube mask were combined (⊙) to generate a mask-combined X-ray image containing more information (see Formula 2).

zᵢ=xᵢ [symbol] mₛ∈{l,t} [Formula 2]

Here, xᵢ is an X-ray image, and mₛ∈{l,t} is a segmented mask. An input z, which is the mask-combined X-ray image, greatly improves the classification deep learning module's understanding of the anatomical structure and the nasogastric tube mask in the X-ray image.

The classification deep learning module used the mask-combined X-ray image as input to train a model gθ to predict whether the nasogastric tube position was abnormal. The input z, which is the mask-combined X-ray image, was input to the model gθ to calculate the probabilities p of a malposition of the nasogastric tube. This is expressed as pᵢ=σ(gθ(zᵢ)), where σ is an activation function. These probabilities are optimized with a cross-entropy loss function, enabling the model to classify positions as either ‘complete’ or ‘incomplete’.

Here, an ResNet-based architecture, which is one of the most widely used neural networks in image analysis, was used as the model gθ. For more accurate performance, a pre-trained model of MedCLIP, which is a state-of-the-art medical vision-text pre-training model, was also used as the classification network gθ.

In addition, a visualization module and a validation module were introduced to increase the interpretability and understand the decision-making process of prediction of the model, and increase reliability through validation. Gradient-weighted Class Activation Mapping (Grad-CAM) was used as the visualization module. Grad-CAM provides a visual explanation of the model's decisions and highlights specific areas of the X-ray image that the model focuses on when determining positioning of the nasogastric tube. This approach not only aids in validating the accuracy and reliability of the model, but also contributing to continuously enhancing the model's performance by identifying potential areas that require additional training or improvement.

All networks and experimental setting were implemented by employing the PyTorch framework. The segmentation deep learning module was trained through five-fold cross-validation to increase robustness, and an ensemble strategy was employed to ensure accurate segmentation results. Due to significant differences in the size of the X-ray images, the input patch size was set to 512×512. Similar implementation details of nnU-Net, which is a state-of-the-art framework based on U-Net architecture, such as optimization methods and data augmentation techniques, were applied. In the classification step, all networks were trained on a single NVIDIA RTX 3090 GPU, and all network parameters were optimized using the SGD optimizer through stratified five-fold cross-validation. A learning rate of a feature extractor of the classification model was set to 0.0005, which is 10 times lower than a learning rate of a classifier consisting of a single MLP layer. All networks were trained for 100 epochs with a batch size of 16, and the weights of the feature extractor were initialized with ResNet-50 trained with MedClip, and the weights of the classifier were initialized randomly. The inputs of all models were uniformly resized to 512×512, and random horizontal flipping was applied with a probability of 0.5 for training. The training dataset was split into training data (80%) and validation data (20%), and all hyperparameters were tuned in the validation phase.

1,799 images were used in the training dataset and 450 images were used in the testing dataset. During the segmentation phase, the training dataset was used for 5-fold cross-validation, and the test set was used to evaluate the model performance. In addition, 378 images provided by Kangwon National University Hospital were used for external validation to further evaluate the robustness and generalizability of the model.

Since the present invention includes two deep learning models for segmentation and classification, six metrics were used to evaluate each model.

To evaluate the segmentation deep learning module, the Dice coefficient and Jaccard coefficient metrics, which indicate similarities between the predictions and ground truth, were first used. These metrics are calculated using true positive, false positive, and false negative (respectively denoted as TP, FP, and FN).

Dice=2TP/(2TP+FP+FN)

Jaccard=TP/(TP+FP+FN) [Formula 3]

Considering imbalanced class distribution, a macro F1-score, balanced accuracy B-ACC, area under a receiver operating characteristic curve (AUC-ROC), and area under a precision-recall curve (AUC-PR) were also evaluated for the evaluation of the classification model. The F1-score and B-ACC are calculated as follows.

F1-score=2×(Precision×Recall)/(Precision+Recall)

Balanced accuracy=(Sensitivity+Specificity)/2 [Formula 4]

An exponential moving average (EMA) was used to track the performance metrics of the segmentation deep learning module. For robust and accurate prediction, the logic of trained models in five-folds was ensembled when evaluating the testing dataset. The segmentation deep learning module achieved a Dice coefficient of 65.35% and a Jaccard coefficient of 57.49% on the testing dataset. (See Table 2)

TABLE 2

| Methods | Metrics | Tube | Tip | Mean |
|---|---|---|---|---|
| nnU-Net | Dice | 91.52 ± 0.14 | 39.16 ± 0.45 | 65.35 ± 0.25 |
| | Jaccard | 85.63 ± 0.22 | 29.37 ± 0.31 | 57.49 ± 0.21 |

As seen in Table 3, when using X-ray images combined with intubation tube masks (lines or tips) as input, a classification ability of the classification deep learning module was consistently enhanced in all metrics such as F1-score, B-ACC, AUC-ROC, and AUC-PR. Compared to the method using only the original X-ray images, the combination of X-ray images and line masks (x+ml) showed performance improvements by 1.74%, 3.71%, 1.79%, and 2.09%, respectively. Similarly, the combination of X-ray images and all masks (x+ml+mt) showed performance improvements by 0.66%, 2.14%, 1.55%, and 1.5%, respectively. Although all combinations of the masks helped improve the model's performance, when all masks (x+ml+mt) were used, performance lowered by 1.08%, 1.57%, 0.24%, and 0.59%, respectively, was shown compared to when the line mask was used alone.

TABLE 3

| Methods | F1-score | B-ACC | AUC-ROC | AUC-PR |
|---|---|---|---|---|
| x | 96.09 ± 1.02 | 95.12 ± 2.17 | 98.17 ± 1.04 | 97.80 ± 0.61 |
| x + ml | 97.83 ± 1.09 | 98.83 ± 0.66 | 99.96 ± 0.03 | 99.89 ± 0.11 |
| x + ml + mt | 96.75 ± 1.31 | 97.26 ± 0.31 | 99.72 ± 0.14 | 99.30 ± 0.33 |

As shown in Table 4, ResNet-50 outperformed other architectures except for ResNet-50 pre-trained with MedCLIP. From these results, it can be confirmed that ResNet-50 is the most suitable model. The performance of ResNet-50 pre-trained with MedCLIP was improved by 2.11%, 1.79%, 0.38%, and 0.85%, respectively, compared to the vanilla ResNet-50. These results suggest the possibility of detecting incorrect insertions of NGT using deep learning-based methods.

TABLE 4

| Methods | F1-score | B-ACC | AUC-ROC | AUC-PR |
|---|---|---|---|---|
| ResNet-18 | 94.36 ± 7.20 | 96.27 ± 4.64 | 97.98 ± 3.85 | 93.61 ± 12.09 |
| ResNet-34 | 93.95 ± 7.51 | 95.44 ± 5.09 | 97.78 ± 3.62 | 93.38 ± 11.82 |
| ResNet-50 | 95.72 ± 3.98 | 97.04 ± 1.61 | 99.57 ± 0.35 | 99.04 ± 0.85 |
| ResNet-50 (pre-trained | 97.83 ± 1.09 | 98.83 ± 0.66 | 99.96 ± 0.03 | 99.89 ± 0.11 |
| w/MedCLIP) | | | | |

As shown in Table 5 below, the present invention achieved Dice Similarity Coefficient of 65.35±0.25, AUC of 99.72, and AUPRC of 99.30. Among these, AUC is an important metric for evaluating the prediction performance of the model, and in particular, it indicates how well the model distinguishes between positive and negative classes. A high AUC value means that the model can distinguish between the normal position and the malposition with high accuracy. In the case of comparative examples, the AUC was only at the maximum level of 92, whereas in the present invention, it achieved as high as 99.72, showing that the reliability was significantly high.

TABLE 5

| | | | | Dice | | |
|---|---|---|---|---|---|---|
| | | Segmen- | | Similarity | | |
| | | tation | Classification | Coefficient | | |
| | Model | Approach | Approach | (%) | AUC | AUPRC |
| Example | Proposed Model | nnU-Net | ResNet-50 (Pre- | 65.35 ± 0.25 | 99.72 | 99.3 |
| | | | trained with | | | |
| | | | MedClip) | | | |
| Comparative | Johnson et al., 2021, ‘Application | None | Inception V3 | N/A | 87 | N/A |
| example 1 | of Convolutional Neural Networks | | (CNN) | | | |
| | for Detecting Critical Malpositions | | | | | |
| | of Enteric Feeding Tubes in | | | | | |
| | Radiographs’ | | | | | |
| Comparative | Mallon et al., 2023, ‘Deep Learning | None | DenseNet-121 | N/A | 90-92 | N/A |
| example 2 | Model Based on DenseNet-121 for | | | | | |
| | Nasogastric Tube Placement | | | | | |
| | Verification in Radiographs’ | | | | | |
| Comparative | Smith et al., 2022, ‘Novel CNN for | None | Custom CNN | N/A | 76 | N/A |
| example 3 | Assessing Enteral Feeding Tube | | (with pre- | | | |
| | Positioning Using Radiographs with | | training) | | | |
| | Pre-training Strategy’ | | | | | |
| Comparative | Drozdov et al., 2023, ‘Deep | None | Custom CNN | N/A | 90 | N/A |
| example 4 | Learning Model to Assist Junior | | | | | |
| | Physicians in Safer Feeding | | | | | |
| | Decisions: Validation and Clinical | | | | | |
| | Impact’ | | | | | |

In addition, 5-fold crossover models were evaluated using the ensemble method for external datasets, and the results are shown in Table 6. The F1-score, B-ACC, AUC-ROC, and AUC-PR values were 70.70%, 82.41%, 88.14%, and 79.77%, respectively. This means that the model can be applied in a wider range of clinical environments.

TABLE 6

| Methods | F1-score | B-ACC | AUC-ROC | AUC-PR |
|---|---|---|---|---|
| ResNet-50 (pre-trained | 70.70 | 82.41 | 88.14 | 79.77 |
| w/MedCLIP) | | | | |

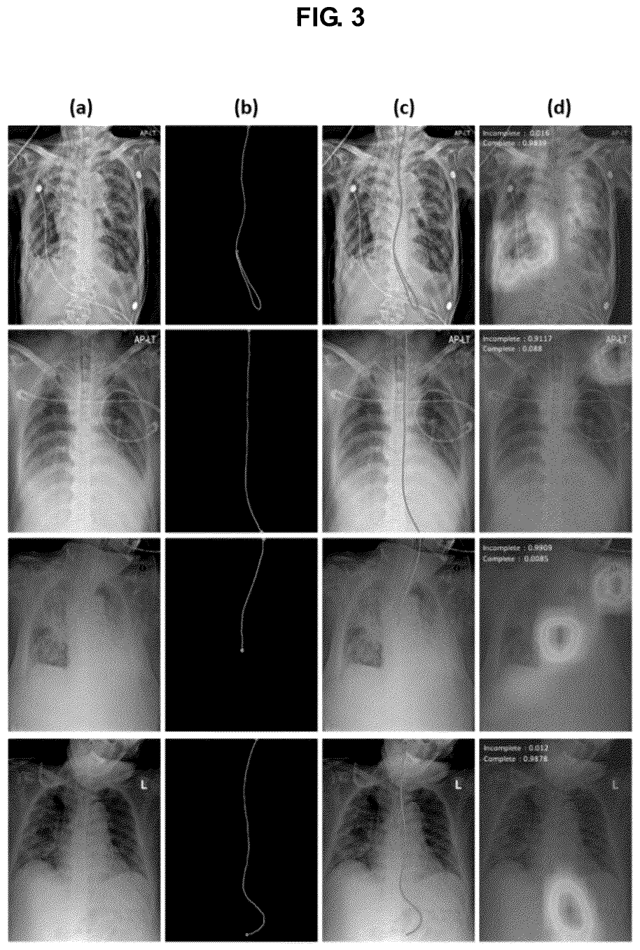

Next, visualization was performed. FIG. 3 is a reference diagram for explaining a process of detecting and analyzing the position of the intubation tube within the human body from an external validation dataset by the system for detecting and analyzing the position of the intubation tube within the human body according to an embodiment of the present invention. FIG. 3 shows (a) an X-ray image, (b) an intubation tube mask labeled with a correct answer learned by the segmentation deep learning module, (c) a result of combining the intubation tube mask generated by the segmentation deep learning module and the original X-ray image, and (d) a heat map result.

Referring to FIG. 3, upper two rows represent incorrectly predicted cases (incomplete, complete), and lower two rows represent correctly predicted cases (incomplete, complete). That is, as seen in the heat map in the upper two rows, there are cases where the classification deep learning module highlights areas unrelated to the intubation tube, such as inappropriate text or peripheral areas. In this case, the validation model outputs the determination result of the classification deep learning module as inappropriate because the area of evidence and the intubation tube mask do not overlap.

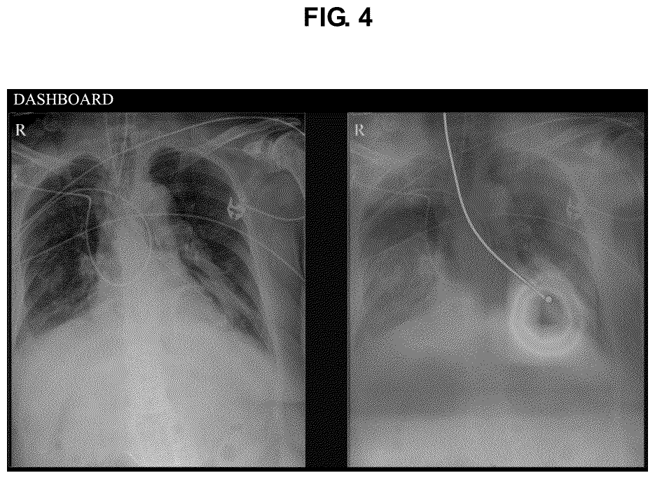

Finally, FIG. 4 is a prototype user interface of a system for detecting and analyzing the position of the intubation tube within the human body according to an embodiment of the present invention. As shown in FIG. 4, if medical staff inputs a relevant X-ray image when positioning analysis of the intubation tube within the human body are required, the position of the intubation tube and the area of evidence for the determination are displayed on the X-ray image and output, and the appropriateness of intubation and the validation result are provided together. The medical staff can visually check whether intubation is appropriate or inappropriate beyond simply being provided with a probability that intubation is appropriate or not, which can assist in clinical determination.

The system for detecting and analyzing the position of the intubation tube within the human body as described above can be implemented as a program (or application) including an executable algorithm that may be executed on a computer. The program may be provided by being stored in a non-transitory computer readable medium. Here, the non-transitory readable medium means a medium that stores data semi-permanently and can be read by a device, rather than a medium that stores data for a short period of time, such as a register, a cache, or a memory. Specifically, the various applications or programs described above may be provided by being stored in non-transitory readable media, such as CDs, DVDs, hard disks, Bu-ray disks, USB, memory cards, ROMs, etc.

The scope of protection of the present invention is not limited to the description and expression of the embodiments explicitly described above. In addition, it is added once again that the scope of protection of the present invention are not limited due to obvious changes or substitutions in the technical field to which the present invention belongs.

This invention was supported by the following National Research and Development Program of the Republic of Korea.
[Project Number] S1402-23-1001
[Ministry Name] NIPA
[Project Management (Specialized) Agency Name] KCA
[Research Task Name] AI-based Medical Solution Commercialization Demonstration Support Task

### [Research Project Name]

Development of Medical AI Software to Improve Patient Safety through Detection and Evaluation of Types and Positions of Tubes and Catheters in Chest X-ray Images
[Contribution Rate] 1/1
[Project Execution Institution Name] Geovision Co., Ltd.
[Research Period] Jul. 1, 2023-Dec. 31, 2025

1. A system for detecting and analyzing a position of an intubation tube within a human body, comprising:
a segmentation deep learning module that receives an X-ray image and generates an intubation tube mask for a position of an intubation tube intubation within a human body included in the X-ray image;
a concatenation module that connects the intubation tube mask and the X-ray image to generate a mask-combined X-ray image; and
a classification deep learning module that receives the mask-combined X-ray image and classifies appropriateness of the position of the intubation tube within the human body.

2. The system of claim 1, wherein the intubation tube is at least one selected from a group consisting of a nasogastric tube, a central venous catheter, an endotracheal tube, and a thoracic drainage tube.

3. The system of claim 1, wherein the intubation tube mask is a mask for a shape and position of a line of the intubation tube.

4. The system of claim 3, wherein the intubation tube mask further includes a mask for a shape and position of a tip of the intubation tube.

5. The system of claim 1, wherein the mask-combined X-ray image is generated by stacking the intubation tube mask and the above X-ray image in a channel home.

6. The system of claim 1, further comprising:
a visualization module that visualizes a determination result of the classification deep learning module, wherein
the visualization module generates a heat-map indicating a area of evidence considered important as a basis for the determination of the classification deep learning module and displays the heat-map by overlapping the heat-map on the mask-combined X-ray image.

7. The system of claim 6, further comprising:
a validation model that validates an appropriateness of the determination result of the classification deep learning module, wherein
the validation model outputs the determination result of the classification deep learning module as inappropriate when the area of evidence and the intubation tube mask do not overlap.
